(19) United States
(12) Patent Application Publication
(10) Pub. No.: US 2024/0115285 A1
(43) Pub. Date: Apr. 11, 2024

(54) AUTOMATIC SURGICAL SHAVER WINDOW LOCK SYSTEM AND METHOD

(71) Applicant: ARTHREX, INC., Naples, FL (US)

(72) Inventors: Robert Fugerer, Lutz, FL (US); Jefferey Slater, Naples, FL (US); Troy Garrison, Fort Myers, FL (US)

(21) Appl. No.: 18/375,401

(22) Filed: Sep. 29, 2023

Related U.S. Application Data
(60) Provisional application No. 63/413,764, filed on Oct. 6, 2022.

(51) Int. Cl. A61B 17/32; A61B 34/20; A61B 90/98

(52) U.S. Cl. CPC A61B 17/32002; A61B 34/20; A61B 90/98; A61B 2017/32004; A61B 2034/2051; A61M 2025/0004

(57) ABSTRACT

A surgical shaver having: an outer tube with at least one outer tube opening at a distal end and an outer hub at a proximal end, the outer hub being configured for attachment to a handpiece; an inner tube rotatably positioned in the outer tube, the inner tube having at least one inner opening at a distal end, a cutting edge at the inner opening for cooperating with the outer tube opening to cut tissue and an inner hub at a proximal end, the inner hub being configured for attachment to a handpiece drive means; an RFID tag affixed to the outer tube or the outer hub, the RFID tag storing information about the shaver; an outer magnet coupled to the outer tube or the outer hub; and an inner magnet coupled to the inner tube or the inner hub.

### CROSS REFERENCE TO RELATED APPLICATION

This application claims priority of U.S. Provisional Patent Application No. 63/413,764, filed on Oct. 6, 2022, entitled AUTOMATIC SURGICAL SHAVER WINDOW LOCK SYSTEM AND METHOD, the entire contents of which is hereby incorporated herein by reference.

### BACKGROUND

The present disclosure relates to surgical devices and, more particularly, to a surgical shaver having an automatic shaver window lock system and method.

Surgical shavers may have an outer elongated tube with a cutting window at a distal end, and an inner elongated tube with a cutting edge at a distal end. The inner elongated tube is moved relative to the outer tube so that the cutting tip cooperates with the cutting window to cut tissue. The shaver may be connected to a suction source to pull tissue into the cutting window and to remove debris from a surgical site. Additionally, the shaver may be connected to a fluid source for irrigating a surgical site. The shaver may be moved between a closed position wherein the cutting tip closes the cutting window and an open position in which the cutting window is open to allow fluid communication with an inside of the inner elongated tube.

A fully open cutting window maximizes flow rate while a fully closed cutting window minimizes flow rate. However, control of the cutting window aperture when entering and exiting a surgical site is important for patient safety to avoid injuring a patient such as by accidentally catching and damaging soft-tissue.

There exists a need for a system and method for automatically controlling a shaver window aperture that remedies the shortcomings of the prior art.

### SUMMARY

This disclosure is directed to systems and methods for automatically detecting and controlling a surgical shaver.

According to an implementation, a method for controlling a rotatable shaver driven by a powered handpiece having a drive means and a sensor, comprises the step of providing a shaver assembly. The shaver assembly has an outer tube with a proximal end, a distal end, at least one outer tube opening at the distal end and an outer hub at the proximal end. The outer hub is configured for attachment of the outer tube to the handpiece. The shaver assembly also has an inner tube rotatably positioned in the outer tube. The inner tube has a proximal end, a distal end, at least one inner opening at the distal end, a cutting edge at the inner opening for cooperating with the outer tube opening to cut tissue and an inner hub at the proximal end. The inner hub is configured for attachment of the inner tube to the drive means. An RFID tag affixed to the outer tube or the outer hub, the RFID tag storing information about the type of shaver assembly. An outer magnet is coupled to the outer tube or the outer hub; and an inner magnet is coupled to the inner tube or the inner hub.

The method further comprises the steps of inserting the shaver assembly into the handpiece; sensing shaver information from the RFID tag; and determining the orientation of the outer tube and the inner tube from at least one of the outer magnet and the inner magnet. The method may further comprise the step of: positioning the inner tube opening at a predetermined position relative to the outer tube opening. Detection of the RFID tag may indicate insertion of the shaver assembly into the handpiece. The RFID may contain information about how many inner openings are present in the shaver assembly. The RFID may contain information about a maximum torque usable with the shaver assembly.

Optionally, the predetermined position of the inner tube opening relative to the outer tube opening is user specific and information about a user's preferred predetermined position is accessible to a controller for controlling the powered handpiece. The step of determining the orientation of the outer tube and the inner tube may further comprise determining the orientation of the outer tube opening by sensing the position of the outer magnet. The step of determining the orientation of the outer tube and the inner tube may further comprise determining the orientation of the at least one inner tube opening by sensing the position of the inner magnet. The step of determining the orientation of the outer tube and the inner tube may further comprise determining the orientation of the at least one inner tube opening by rotating the inner tube relative to the outer tube and sensing changes in the position of the inner magnet. Optionally, the inner tube is rotated relative to the outer tube by about 360 degrees to determine orientation. Optionally, the inner tube is rotated relative to the outer tube until the inner magnet has passed either closest to or farthest from the sensor to determine orientation. The step of determining the orientation of the outer tube and the inner tube may be automatically initiated once detection of the RFID tag indicates insertion of the shaver assembly into the handpiece. The inner tube may have two inner windows.

This disclosure is also directed to a surgical shaver system. A surgical shaver system according to an implementation has a powered handpiece with a motor; a controller for controlling the motor; an RFID sensor coupled to the controller; and a magnet sensor coupled to the controller. A shaver assembly has an outer tube with a proximal end, a distal end, at least one outer tube opening at the distal end and an outer hub at the proximal end, the outer hub being configured for attachment of the outer tube to the handpiece. The shaver assembly also has an inner tube rotatably positioned in the outer tube, the inner tube having a proximal end, a distal end, at least one inner opening at the distal end, a cutting edge at the inner opening for cooperating with the outer tube opening to cut tissue and an inner hub at the proximal end. The inner hub is configured for attachment of the inner tube to the drive means.

An RFID tag affixed to the outer tube or the outer hub, the RFID tag storing information about the type of shaver assembly. An outer magnet is coupled to the outer tube or the outer hub. An inner magnet coupled to the inner tube or the inner hub. The RFID sensor is configured to detect the presence of the RFID tag and to read information from the RFID tag. The controller is configured to operate the motor to rotate the inner tube relative to the outer tube while using the magnet sensor to determine the orientation of the outer tube and the inner tube from at least one of the outer magnet and the inner magnet. The controller may be further configured to position the inner tube opening at a predetermined position relative to the outer tube opening.

The RFID tag may have information about how many inner openings are present in the shaver assembly. The RFID tag may have information about a maximum torque usable with the shaver assembly. The surgical shaver system may also have a memory coupled to the controller, the memory further comprising user preferences for the predetermined position of the at least one inner tube opening relative to the outer tube opening. The inner tube may have two inner windows.

This disclosure is also directed to a surgical shaver. A surgical shaver according to an implementation has an outer tube with a proximal end, a distal end, at least one outer tube opening at the distal end and an outer hub at the proximal end. The outer hub is configured for attachment of the outer tube to a handpiece. The surgical shaver also has an inner tube rotatably positioned in the outer tube, the inner tube having a proximal end, a distal end, at least one inner opening at the distal end, a cutting edge at the inner opening for cooperating with the outer tube opening to cut tissue and an inner hub at the proximal end. The inner hub is configured for attachment of the inner tube to a handpiece drive means.

An RFID tag is affixed to the outer tube or the outer hub, the RFID tag storing information about the type of shaver. An outer magnet is coupled to the outer tube or the outer hub. An inner magnet is coupled to the inner tube or the inner hub.

These and other features are described below.

### BRIEF DESCRIPTION OF THE DRAWINGS

The features, aspects and advantages of the present invention will become better understood with regard to the following description, appended claims and accompanying figures wherein:

FIG. 1 is a schematic diagram of a shaver system having an automatic window control according to an implementation;

FIG. 2 is a schematic diagram of the shaver assembly of FIG. 1 showing the inner shaft and the outer shaft in different orientations;

FIG. 3 is a schematic diagram of a shaver assembly having an automatic window control according to an implementation with an inner shaft with two inner openings;

FIG. 4 is a flow chart illustrating a method for an automatic window control according to an implementation; and

FIG. 5 is a graph illustrating detected magnetic signals as the inner tube is rotated relative to the outer tube according to an implementation.

### DETAILED DESCRIPTION

In the following description of the preferred implementations, reference is made to the accompanying drawings which shows by way of illustration specific implementations in which the invention may be practiced. It is to be understood that other implementations may be utilized and structural and functional changes may be made without departing from the scope of this disclosure.

The present disclosure is directed to a system and method for producing high dynamic range and fluorescent images.

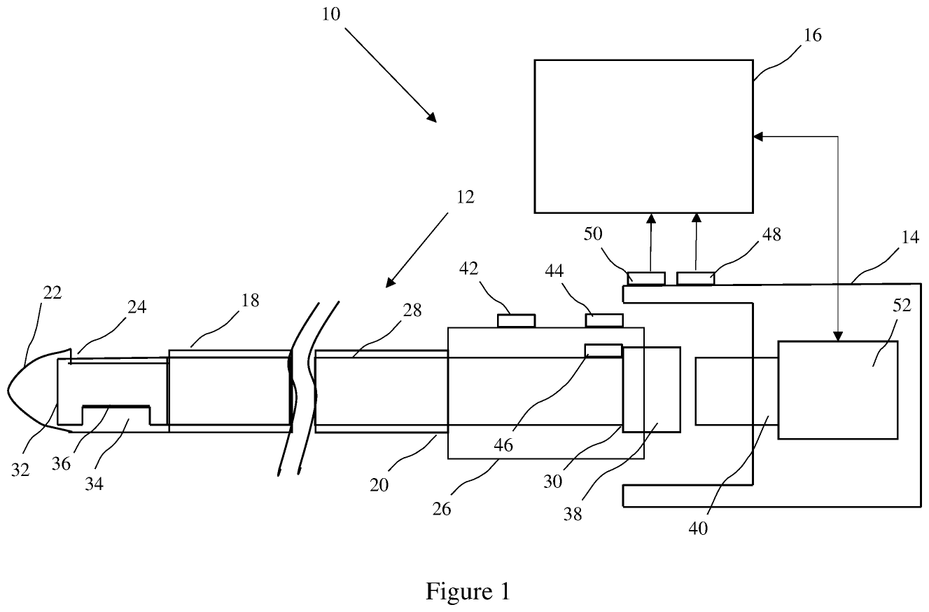

With reference to FIG. 1, a shaver system 10 according to an implementation has a shaver assembly 12, a handpiece 14 and a controller 16. The shaver assembly 12 has an outer tube 18 having a proximal end 20, a distal end 22, at least one outer tube opening 24 at the distal end and an outer hub 26 at the proximal end. The outer hub 26 is configured for attachment of the shaver assembly 12 to the handpiece 14.

The shaver assembly 12 also has an inner tube 28 rotatably positioned in the outer tube 18. The inner tube 28 has a proximal end 30, a distal end 32, at least one inner opening 34 at the distal end and a cutting edge 36 proximal to the inner opening for cooperating with the outer tube opening 24 to cut tissue. The inner tube 28 also has an inner hub 38 at a proximal end, the inner hub being configured for attachment of the inner tube to a drive means 40 in the handpiece 14. The drive means 40 is connected to a motor 52.

A radio frequency identification tag (RFID tag) 42 is coupled to the outer tube 18 or the outer hub 26. The RFID tag 42 stores information about the shaver assembly 12. For example, the RFID tag 42 may store information about the number of inner openings 34 in the shaver assembly 12. Additionally, for example, the RFID tag 42 may store information about a maximum torque usable with the shaver assembly 12; a maximum rotation speed of the shaver assembly, a minimum rotation speed of the shaver assembly, allowable oscillation modes, shaver system authentication data, automatic shaver window locking information, shaver orientation threshold data, optimized algorithm identification for the shaver assembly, and graphic images for displaying shaver assembly orientations.

An outer magnet 44 is coupled to the outer tube 18 or the outer hub 26. The outer magnet 44 is positioned in a predetermined relationship to the at least one outer tube opening 24 so that by sensing the position of the outer magnet a position of the at least one outer tube opening may be known. An inner magnet 46 is coupled to the inner tube 28 or the inner hub 38. The inner magnet 46 is positioned in a predetermined relationship to the at least one inner opening 34 so that by sensing the position of the inner magnet a position of the at least one inner opening may be known.

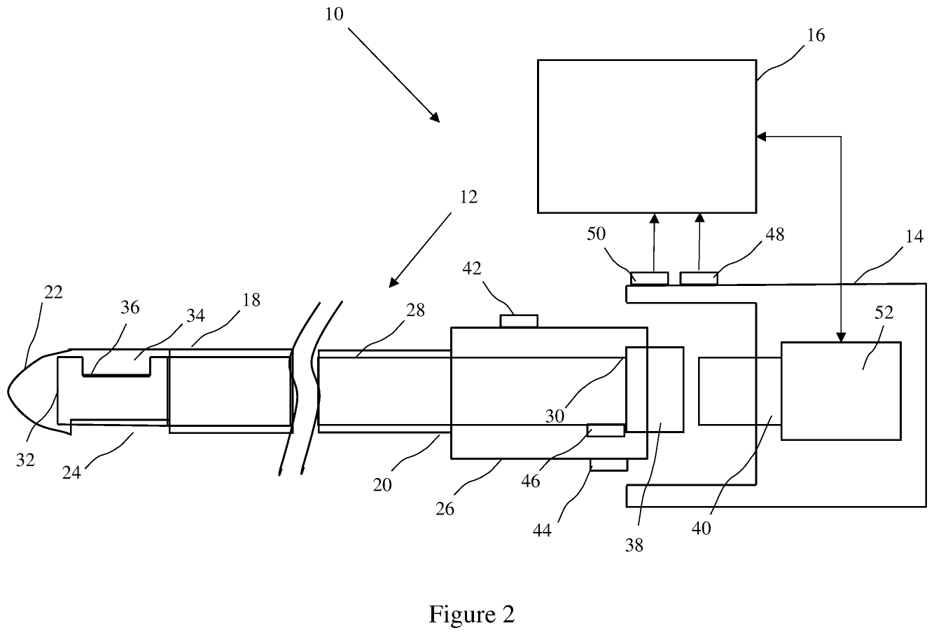

In an implementation, as shown in FIGS. 1 and 2, the inner magnet 46 is positioned at an opposite radial position to the inner opening 34, while the outer magnet 44 is positioned at approximately the same radial position as the outer opening. In an implementation the inner magnet 46 and the outer magnet 44 are oriented in the same direction (for example, with the magnetic north of each magnet pointing outward from the inner tube 28 and the outer tube 18 respectively). The magnetic attraction between the inner magnet 46 and the outer magnet 44 may be usable to hold the inner tube 28 in a preferred orientation relative to the outer tube 18.

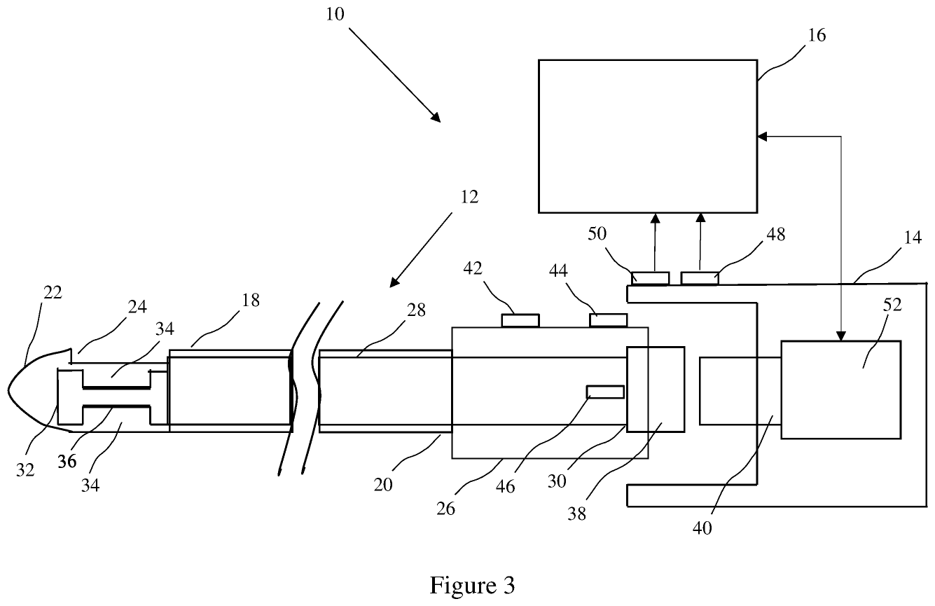

In an additional implementation, as shown in FIG. 3, the inner shaft has two inner openings 34. The inner magnet 46 is positioned radially between the two inner openings 34. For example, the inner openings 34 may be 180 degrees apart and the inner magnet 46 is positioned 90 degrees apart from each of the inner openings.

The handpiece 14 has a magnet sensor 48, such as a hall sensor, for sensing the outer magnet 44 and the inner magnet 46. The handpiece 14 also has an RFID sensor 50 for sensing a presence of the RFID tag 42 and for reading information from the RFID tag. The magnet sensor 48 and the RFID sensor 50 are coupled to the controller 16. In additional implementations, other types of sensor systems may be used instead of magnet sensor 48 in combination with outer magnet 44 and inner magnet 46. For example, optical sensors, audio sensors or vibration sensors may be used.

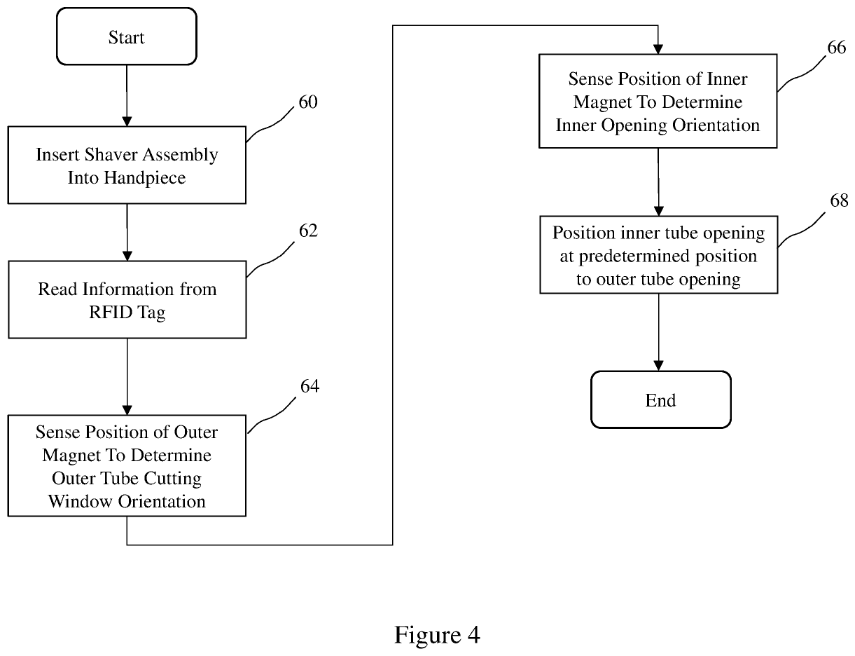

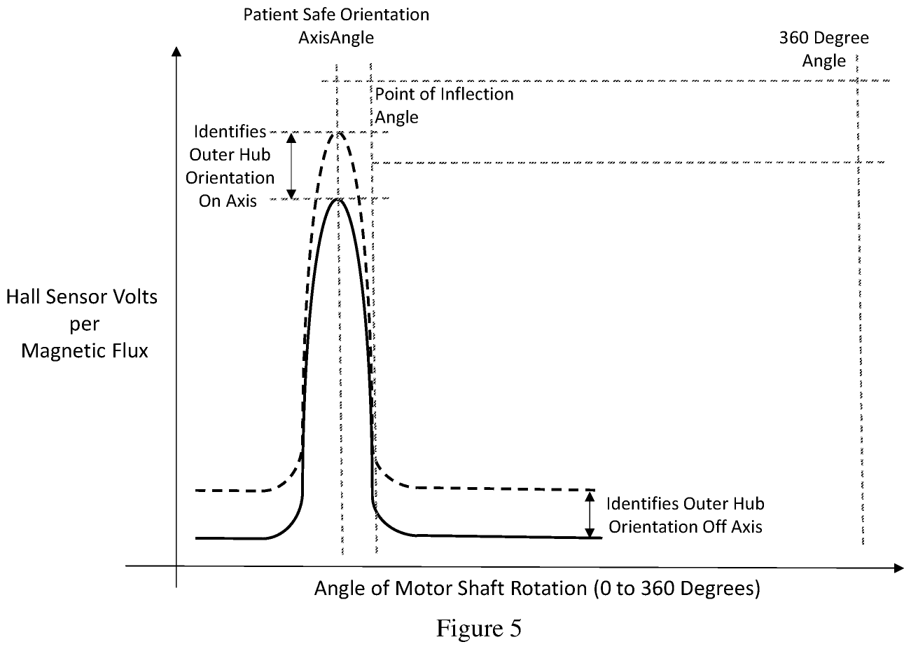

Operation of the shaver system 10 according to an implementation will now be described with reference to FIGS. 4 and 5. The shaver assembly 12 is inserted into the handpiece 14 in step 60. The RFID sensor 50 senses the presence of the RFID tag 42 in the handpiece, reads information from the RFID tag and sends information to the controller 16 in step 62. Detection of the RFID tag signals insertion and removal of the shaver assembly from the handpiece.

As seen in FIGS. 1 and 2, the shaver assembly 12 may be inserted into the handpiece 14 in a plurality of orientations. For example, in a first orientation as shown in FIG. 1, the outer tube opening 24 may be facing up and the inner opening 34 facing down. In a second orientation as shown in FIG. 2, the outer tube opening 24 may be facing down and the inner opening 34 facing up. The first and second orientations illustrate a patient safe aperture-closed orientation.

The magnet sensor 48 detects the position of the outer magnet 44 to determine the orientation of the outer tube opening 24 in step 64. The controller can identify the orientation of the outer tube opening by the presence of a magnet signal if the outer magnet is proximal to the magnet sensor 48 or by the absence of a magnet signal if the outer magnet is positioned away from the magnet sensor (such as 180 degrees away). Step 64 may not be necessary in some situations. For example, the shaver assembly 12 and handpiece 14 may be configured such that the shaver assembly may only be inserted into the handpiece with the outer tube opening 24 in a single known orientation.

The magnet sensor 48 detects the position of the inner sensor to determine the orientation of the inner opening 34 in step 66. In an implementation, the controller 16 slowly rotates the inner tube 28 one revolution and initiates a search algorithm to orient the motor with respect to inner opening 34 position using information from the magnet sensor 48 and the handpiece motor 52. During the rotation, the controller 16 is gathering magnet sensor 48 data and correlating that data with motor position. The rotation may be conducted very slowly to prevent patient injury. The rotation may be conducted at very low torque to prevent patient injury.

As illustrated in FIG. 5, a search algorithm according to an implementation completes after rotating the inner tube 28 one rotation. During this time, the controller 16 gathers all sensor data and correlates that data with motor position. The controller 16 tracks the rotational change between maximum magnet strength in angle of rotation (between 0 and 360 degrees) while the motor 52 rotates the inner tube 28 one revolution. Because the search algorithm only needs to identify a single inner opening 34 position, no signal thresh-holding is required, and the algorithm only needs to scan one revolution while searching for the maximum magnet strength, so no absolute signal levels are required. With this approach, magnet variance is acceptable within a minimum signal-to-noise ratio.

Also with reference to FIG. 5, in an additional implementation, the search algorithm can complete after rotating the inner tube 28 past a point of direction change in magnetic signal strength, referred to as the point of inflection. During this time, the controller 16 gathers magnet sensor 48 data and correlates that data with motor 52 position. The controller tracks the rotational change between maximum magnet strength in angle of rotation (between 0 degrees and the point of inflection in the magnetic strength data) while the motor 52 is rotating the inner tube 28. Because the algorithm only needs to identify a single inner opening 34 position, no signal thresh-holding is required, and the algorithm only needs to scan, in this implementation, to the point of inflection while searching for the maximum magnet strength, so no absolute signal levels are required. With this approach, magnet variance is acceptable within a minimum signal-to-noise ratio, and the time to acquire the maximum magnet strength is minimized.

When the search algorithm is conducted, the controller 16 is provided with information that synchronizes motor shaft position with the specific orientation detected through the magnet sensor 48. If an inserted cutting accessory has the outer hub rotated 180 degrees, then the patient safe aperture-closed orientation occurs when the inner hub is also rotated 180 degrees. In a situation where the outer magnet 44 and the inner magnet 46 are both positioned away from the magnet sensor 48, at least two additional magnets would typically be used to detect orientation. However, the present system uses detection of the RFID tag 42 to eliminate the need for additional magnets. The ability to use just one outer magnet 44 and one inner magnet 46 helps to reduce cost, manufacturing complexity and reliability.

After detecting the RFID tag 42 and detecting the orientation of the outer tube opening 24 and the inner opening 34, the controller 16 controls the motor 52 to place the inner opening in a predetermined position in step 66. The inner opening 34 may be moved into position very quickly after the orientation of the outer tube opening 24 and the inner opening 34 are determined. This allows for real-time tracking and control of a cutting aperture formed by the outer tube opening 24 and the inner opening 34, not only when the inner tube 28 is static, but also when the blade is operating in oscillation modes that may require an accurate number of rotations to be tracked, or a specific aperture percent open or closed when an oscillation mode reverses direction.

The controller may use additional sensors (not shown) in communication with the motor 52 as well as sensorless information gathered from the motor's back-EMF to place the motor into a patient safe closed-aperture position. Additionally, any outer tube opening 24 and inner opening 34 aperture can be set rapidly after orientation is completed. As long as the shaver assembly 12 remains in the handpiece 14, the orientation remains valid and the controller 16 can quickly move the inner opening 34 to change the window aperture to any position using a commutation scheme for tracking motor position. The controller 16 can also stop the motor 52 so that the inner opening 34 creates any window aperture position desired.

In an implementation, as shown in FIG. 3, the shaver assembly 12 is a double cut device where there are two inner openings 34. In this implementation, the controller only needs to move the inner shaft 28 between 90 degree increments to close the aperture formed by the outer tube opening 24 and the inner opening 34. Information about whether the shaver assembly 12 is a single cut device or a double cut device can be stored on the RFID tag 42 so that the controller 16 can control the motor 52 accordingly.

In an implementation, the controller 16 has a memory or is in communication with a memory containing surgeon preferences. The controller 16 can control the motor 52, and in turn the aperture formed by the outer tube opening 24 and the inner opening 34, in accordance with user preferences. For example, some users may prefer that the controller 16 stop the motor 52 to align the outer tube opening 24 with the inner opening 34 so as to allow for suction or irrigation through the aperture. Alternatively, for example, some surgeons may prefer that the controller 16 stop the motor 52 with the outer tube opening 24 out of alignment with the inner opening 34 to prevent suction or irrigation and to prevent a patient's tissue from entering the outer tube opening. Alternatively, for example, some surgeons may prefer that the control 16 stop the motor 52 with the outer tube opening 24 only partially out of alignment with the inner opening 34 to allow for some suction or irrigation while reducing the likelihood of a patient's tissue entering the outer tube opening.

The system and method for automatically controlling a shaver window opening mitigates the risks associated with manual window-lock. The system allows for precise control of a shaver window opening for entering and exiting a surgical site in accordance with user preferences. This is an improvement over systems where a user must repeatedly manually activate the motor, and deactivate the motor while monitoring the cutting window until it is in a desired position. The system enables users to set up profiles prior to use, which guarantees the desired window lock behavior, creating a “set-it and forget-it” system that reduces patient risk and human error.

Additionally, the system and methods taught herein allow for the use of an RFID tag and a single outer magnet and a single inner magnet to automatically identify outer tube orientation and cutting edge orientation and position without user intervention. The use of a small number of magnets help avoid issues with magnet strength degradation or magnet sensor tolerances. The system and methods taught herein also eliminate the need for continuous magnet re-orientation after a shaver assembly has been inserted into the handpiece.

There is disclosed in the above description and the drawings, a system and method for automatically controlling a shaver window opening that fully and effectively overcomes the disadvantages associated with the prior art. However, it will be apparent that variations and modifications of the disclosed implementations may be made without departing from the principles of the invention. The presentation of the implementations herein is offered by way of example only and not limitation, with a true scope and spirit of the invention being indicated by the following claims.

Any element in a claim that does not explicitly state “means” for performing a specified function or “step” for performing a specified function, should not be interpreted as a “means” or “step” clause as specified in 35 U.S.C. § 112.

What is claimed is:

1. A method for controlling a rotatable shaver driven by a powered handpiece having a drive means and a sensor, the method comprising the steps of:
providing a shaver assembly comprising:
an outer tube having a proximal end, a distal end, at least one outer tube opening at the distal end and an outer hub at the proximal end, the outer hub being configured for attachment of the outer tube to the handpiece;
an inner tube rotatably positioned in the outer tube, the inner tube having a proximal end, a distal end, at least one inner opening at the distal end, a cutting edge at the inner opening for cooperating with the outer tube opening to cut tissue and an inner hub at the proximal end, the inner hub being configured for attachment of the inner tube to the drive means;
an RFID tag affixed to the outer tube or the outer hub, the RFID tag storing information about the type of shaver assembly;
an outer magnet coupled to the outer tube or the outer hub; and
an inner magnet coupled to the inner tube or the inner hub;
inserting the shaver assembly into the handpiece;
sensing shaver information from the RFID tag; and
determining the orientation of the outer tube and the inner tube from at least one of the outer magnet and the inner magnet.

2. The method of claim 1 further comprising the step of positioning the inner tube opening at a predetermined position relative to the outer tube opening.

3. The method of claim 1 wherein detection of the RFID tag indicates insertion of the shaver assembly into the handpiece.

4. The method of claim 1 wherein the RFID contains information about how many inner openings are present in the shaver assembly.

5. The method of claim 1 wherein the RFID contains information about a maximum torque usable with the shaver assembly.

6. The method of claim 1 wherein the predetermined position of the inner tube opening relative to the outer tube opening is user specific and information about a user's preferred predetermined position is accessible to a controller for controlling the powered handpiece.

7. The method of claim 1 wherein the step of determining the orientation of the outer tube and the inner tube further comprises determining the orientation of the outer tube opening by sensing the position of the outer magnet.

8. The method of claim 1 wherein the step of determining the orientation of the outer tube and the inner tube further comprises determining the orientation of the at least one inner tube opening by sensing the position of the inner magnet.

9. The method of claim 1 wherein the step of determining the orientation of the outer tube and the inner tube further comprises determining the orientation of the at least one inner tube opening by rotating the inner tube relative to the outer tube and sensing changes in the position of the inner magnet.

10. The method of claim 9 wherein the inner tube is rotated relative to the outer tube by about 360 degrees to determine orientation.

11. The method of claim 9 wherein the inner tube is rotated relative to the outer tube until the inner magnet has passed either closest to or farthest from the sensor to determine orientation.

12. The method of claim 9 wherein the step of determining the orientation of the outer tube and the inner tube is automatically initiated once detection of the RFID tag indicates insertion of the shaver assembly into the handpiece.

13. The method of claim 1 wherein the inner tube further comprises two inner windows.

14. A surgical shaver system comprising:
a powered handpiece further comprising:
a motor;
a controller for controlling the motor;
an RFID sensor coupled to the controller; and
a magnet sensor coupled to the controller;
a shaver assembly comprising:
an outer tube further comprising a proximal end, a distal end, at least one outer tube opening at the distal end and an outer hub at the proximal end, the outer hub being configured for attachment of the outer tube to the handpiece;
an inner tube rotatably positioned in the outer tube, the inner tube having a proximal end, a distal end, at least one inner opening at the distal end, a cutting edge at the inner opening for cooperating with the outer tube opening to cut tissue and an inner hub at the proximal end, the inner hub being configured for attachment of the inner tube to the drive means;
an RFID tag affixed to the outer tube or the outer hub, the RFID tag storing information about the type of shaver assembly;
an outer magnet coupled to the outer tube or the outer hub; and
an inner magnet coupled to the inner tube or the inner hub;
wherein the RFID sensor is configured to detect the presence of the RFID tag and to read information from the RFID tag; and
wherein the controller is configured to operate the motor to rotate the inner tube relative to the outer tube while using the magnet sensor to determine the orientation of the outer tube and the inner tube from at least one of the outer magnet and the inner magnet.

15. The surgical shaver system of claim 14 wherein the controller is further configured to position the inner tube opening at a predetermined position relative to the outer tube opening.

16. The surgical shaver system of claim 14 wherein the RFID tag further comprises information about how many inner openings are present in the shaver assembly.

17. The surgical shaver system of claim 14 wherein the RFID tag further comprises information about a maximum torque usable with the shaver assembly.

18. The surgical shaver system of claim 14 further comprising a memory coupled to the controller, the memory further comprising user preferences for the predetermined position of the at least one inner tube opening relative to the outer tube opening.

19. The surgical shaver system of claim 14 wherein the inner tube further comprises two inner windows.

20. A surgical shaver comprising:
an outer tube further comprising a proximal end, a distal end, at least one outer tube opening at the distal end and an outer hub at the proximal end, the outer hub being configured for attachment of the outer tube to a handpiece;
an inner tube rotatably positioned in the outer tube, the inner tube having a proximal end, a distal end, at least one inner opening at the distal end, a cutting edge at the inner opening for cooperating with the outer tube opening to cut tissue and an inner hub at the proximal end, the inner hub being configured for attachment of the inner tube to a handpiece drive means;
an RFID tag affixed to the outer tube or the outer hub, the RFID tag storing information about the type of shaver;
an outer magnet coupled to the outer tube or the outer hub; and
an inner magnet coupled to the inner tube or the inner hub.
